(19) United States
(12) Patent Application Publication
(10) Pub. No.: US 2005/0043672 A1
(43) Pub. Date: Feb. 24, 2005

(54) Multi-part ozone treatment chamber

(76) Inventors: Vladimir Piuk, Kiryat Yam (IL); Mark Shnaiderman, Kiryat Yam (IL); Oren Malkin, Tal Shahar (IL)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/496,136

(22) PCT Filed: Nov. 20, 2001
(86) PCT No.: PCT/IL01/01069

(51) Int. Cl. A61M 37/00

(52) U.S. Cl. 604/23

(57) ABSTRACT

A multi-part treatment chamber may have a first piece and a second piece, where either the first or second piece may have a treatment agent inlet and outlet, or each piece may have either an inlet or an outlet. The first and second pieces are configured such that they may be connected to one another to form a chamber having an inlet, an outlet, and an opening through which the tissue to be treated may enter the chamber.

### FIELD OF THE INVENTION

The present invention relates generally to the field of treatment chambers. More specifically, the present invention relates to a multi-part ozone treatment chamber.

### BACKGROUND OF THE INVENTION

Treatment chambers within which living tissue may be treated with a treatment agent (e.g. oxygen, ozone, etc.) are used today. These chambers commonly have an opening through which the tissue to be treated is inserted and an inlet and outlet through which a treatment agent may enter and leave the chamber. Treatment chamber of the prior art are commonly made from metal and/or glass and not meant to be disposable. Further more, they are constructed of one piece and thus cumbersome to manipulate and operate.

### BRIEF DESCRIPTION OF THE DRAWINGS

As part of the present invention, there is a multi-part treatment chamber having a first piece and a second piece. Either the first or second piece may have a treatment agent inlet and outlet, or each piece may have either an inlet or an outlet. The first and second pieces are configured such that they may be connected to one another to form a chamber having an inlet, an outlet, and an opening through which the tissue to be treated may enter the chamber. There may also be a sealing agent on the contact surfaces between the first and second pieces, and a sealing material around the chamber's opening.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

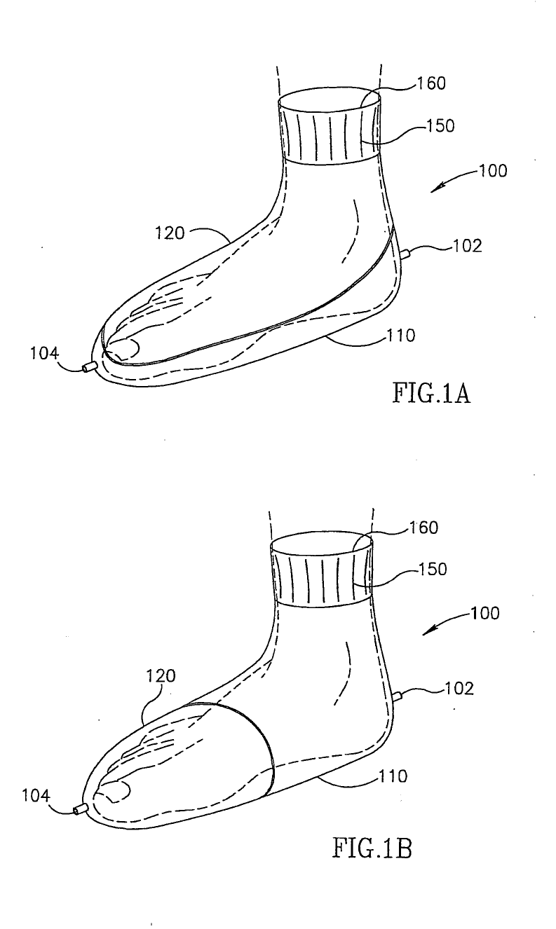

FIG. 1A shows a multi-part treatment chamber for a human foot according to the present invention;

FIG. 1B shows a second embodiment of a multi-part treatment chamber for a human foot according to the present invention;

FIG. 1C shows a third embodiment of a multi-part treatment chamber for a human foot according to the present invention;

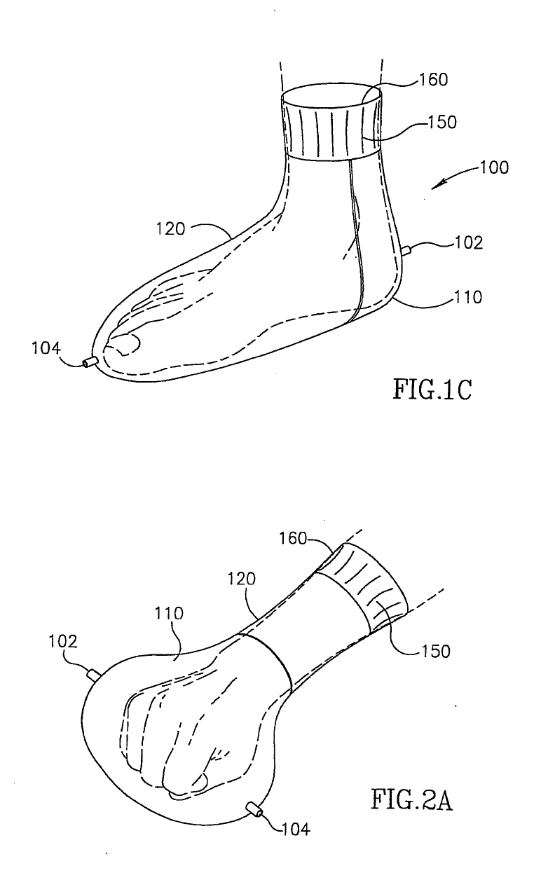

FIG. 2A shows a multi-part treatment chamber for a human hand or arm according to the present invention;

FIG. 2B shows a second embodiment of a multi-part treatment chamber for a human hand or arm according to the present invention;

FIG. 2C shows a third embodiment of a multi-part treatment chamber for a human hand or arm according to the present invention;

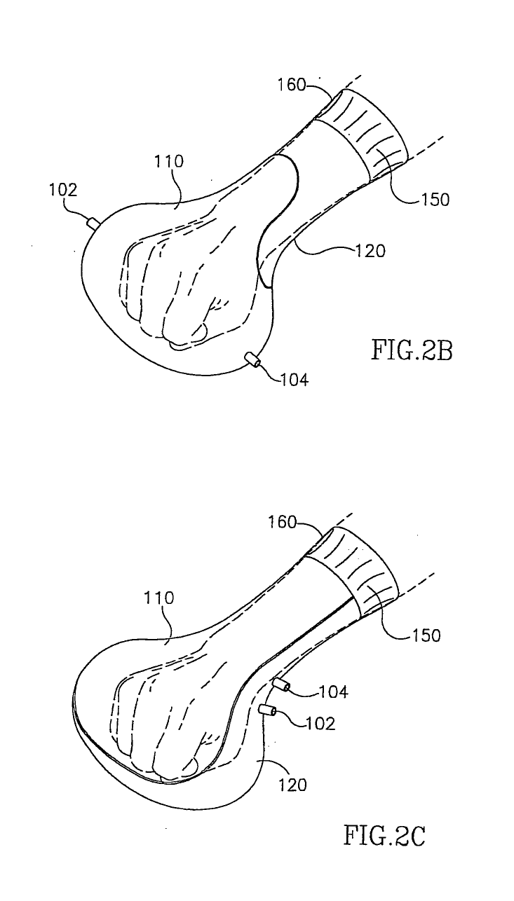

FIG. 3 shows a close-up of the contact surfaces between the first and second chamber pieces; and

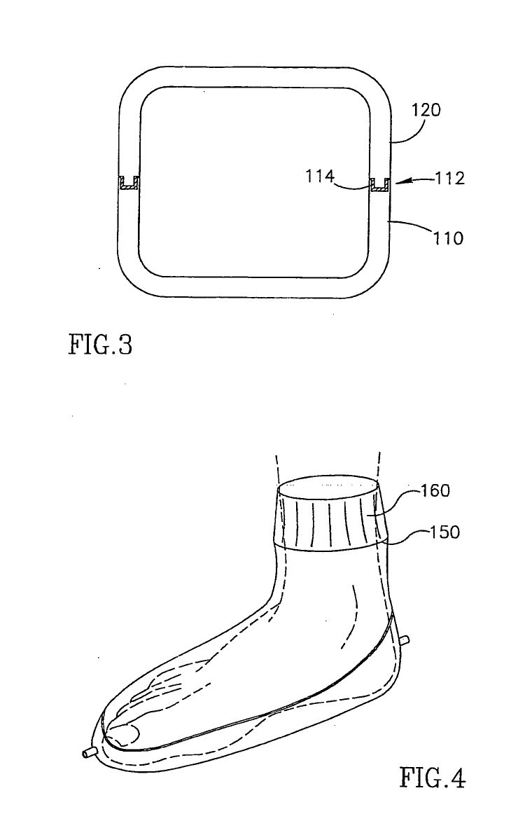

FIG. 4 shows a close up of the sealing material at the opening of the multi-part chamber in FIG. 1.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION

As part of the present invention, there is a multi-part treatment chamber having a first piece and a second piece. Either the first or second is piece may have a treatment agent inlet and outlet, or each piece may have either an inlet or an outlet. The first and second pieces are configured such that they may be connected to one another to form a chamber having an inlet, an outlet, and an opening through which the tissue to be treated may enter the chamber. There may also be a sealing agent on the contact surfaces between the first and second pieces, and a sealing material around the chamber's opening. The contact surfaces of one of the pieces may form a concave channel into which the contact surfaces of the other piece may fit.

Turing now to FIG. 1A, there is shown a foot shaped treatment chamber 100 according to the present invention. The treatment chamber has a first piece 110, which in this case forms the bottom of the chamber, and a second piece 120 forming the top of the chamber. An opening in the second piece 120 forms the chamber's opening 120, and a sealing material 160 may be attached to the periphery of the opening 150. The sealing material 160 may have the shape of a sleeve or sheet and may be composed of either silicon, rubber, polyurethane, poxy or plastic. FIG. 4 illustrates how a sealing material may be used to form a seal around a limb placed in to the chamber through opening 150. The sealing material may form a seal between the chamber and the limb such that little or no treatment agent may escape through the opening 150.

Turning now to FIGS. 1B and 1C, there are illustrated two further embodiments of a foot shaped treatment chamber 100 according to the present invention. In FIG. 1B, the first piece 110 of the chamber 100 forms the heal portion of the chamber and the second piece 120 forms the toe or ball section of the chamber 100. While in FIG. 1C, the first piece 110 and the second piece 120 together form the back or heal portion of the chamber 100.

As shown and suggested through FIGS. 1A to 1C, the exact location of the inlet 102 and outlet 104 is not relevant Furthermore, the designation “first piece” and “second piece” are interchangeable, and the exact portion of the chamber 100 which each forms is also not limited.

Turning now to FIGS. 2A to 2C, there are illustrated three embodiments of a treatment chamber 100 for in the shape of an arm. The various elements of the chambers 100 in FIGS. 2A to 2C are the same as describer above for FIGS. 1A to 1C. Only the specific shapes of the element may be adjusted to accommodate an arm as opposed to a foot.

Turning now to FIG. 3, there is shown a cross section of the chamber of FIG. 1A, with a close up of the contact surfaces between the first piece 110 and second piece 120. The contact surfaces of the first piece 110 are shaped such as to form a concave channel 112. The contact surfaces of the second piece 120 are shaped to fit into the concave channel 112. A sealing agent 114 may be used to connect the first piece 110 and the second piece 120. The sealing agent may be composed of either silicon, rubber, polyurethane, poxy or plastic.

Both pieces of the chamber 100 may be composed of one or more materials suitable for the particular treatment agent for which the chamber 100 is to be used. The materials which may be considered for use in either piece of the chamber when the treatment agent is ozone include, but are not limited to, plastics, silicon, metal, and treated rubber.

While certain features of the present invention have been illustrated and described herein, many modifications, substitutions, changes, and equivalents will now occur to those skilled in the art. It is, therefore, to be understood that the appended claims are intended to cover all such modifications and changes as fall within the true spirit of the invention.

1. A treatment chamber comprised of a first piece and a second piece, each piece having a contact area such that the contact areas of said first and second pieces are in contact with one another through a sealing agent; a treatment agent inlet connected to said first piece; and a treatment agent outlet connected to either said first or second pieces.

2. The treatment chamber according to claim 1, wherein the sealing agent is selected from the group consisting of rubber, glue, silicon, natural resin, synthetic resin, polyurethane, and poxy.

3. The treatment chamber according to claim 2, wherein said first piece is comprised of a material selected from the group consisting of silicon, polyurethane, poxy, metal, and plastic.

4. The treatment chamber according to claim 3, further comprising a sealing material affixed near an opening of said chamber.

5. The treatment chamber according to claim 4, wherein the sealing material is selected from a group consisting of silicon, rubber, polyurethane, poxy and plastic.

6. The treatment chamber according to claim 3, wherein the chamber is hollow and is of a shape accommodating a limb.

7. The treatment chamber according to claim 6, wherein the shape of the treatment chamber would accommodate a limb selected from the group consisting of a foot, a leg, an arm, and a hand.

8. The treatment chamber according to claim 3, wherein the chamber is hollow and has a sealing material configured for attachment to a portion of a torso.

9. The treatment chamber according to claim 1, wherein the contact surfaces of said first piece form a concave channel and the contact surfaces of said second piece fit into the concave channel.

10. The treatment chamber according to claim 9, wherein said sealing agent is within the concave channel.
